(19) United States
(12) Patent Application Publication
(10) Pub. No.: US 2011/0104730 A1
(43) Pub. Date: May 5, 2011

(54) MESOSCALE BIOREACTOR PLATFORM FOR PERFUSION

(75) Inventors: Jacob Mollenbach Larsen, Copenhagen (DK); Ulrich Kruhne, Copenhagen (DK)

(73) Assignee: SMART BIOSYSTEMS APS, Copenhagen NV (DK)

(21) Appl. No.: 12/674,762

(22) PCT Filed: Aug. 1, 2008
(86) PCT No.: PCT/DK08/50192

§ 371 Date: Jan. 3, 2011

(30) Foreign Application Priority Data
Aug. 24, 2007 (DK) PA 2007 01213

(51) Int. Cl. C12M 1/00; C12M 1/34; C12M 1/38; C12M 1/36; C12N 5/071; C12Q 1/02; C12M 3/00; C12M 1/04; C12M 1/02; C12N 5/078; C12N 5/075; C12N 5/0783; C12N 5/0784; C12N 5/0786

(52) U.S. Cl. 435/29; 435/289.1; 435/287.1; 435/287.2; 435/288.7; 435/286.1; 435/286.5; 435/286.6; 435/325; 435/243

(57) ABSTRACT

Disclosed is a mesoscale bioreactor platform including two or more liquid reservoirs in fluid communication with a culture chamber which chamber is in fluid communication with an exit. The platform allows the chamber to be perfused with a flow of liquid from one or more of the liquid reservoirs. The integrated reservoirs for liquids remove the need for external supplies of liquid to the culture chamber during cell culture experiments requiring perfusion of liquids. Moreover, with two or more reservoir it is possible to supply the culture chamber with different types of liquids.

### FIELD OF THE INVENTION

This invention relates to a mesoscale bioreactor platform and a control unit for the bioreactor platform as well as a system comprising the mesoscale bioreactor platform and the control unit for the bioreactor platform. The mesoscale bioreactor platform is suited for culturing biological cells, it is especially suited for culturing mammalian cells, such as embryos or stem cells. More particularly it is suited for use in in vitro fertilisation procedures.

### PRIOR ART

The procedures currently employed in in vitro fertilisation for embryo culture rely on culturing the embryos in Petri-dishes under static conditions. Such methodology is labour-intensive, as changes of growth media require a large degree of manual handling. Manual handling always introduces a risk of contamination, and moreover the static conditions do not provide much resemblance with in vivo conditions, as it is difficult to meet the changing needs of an embryo. In contrast to the current-day in vitro static conditions an embryo in vivo is exposed to a constantly changing environment, and the requirements of an embryo in one stage of development may be very different to those in another stage of development. The conditions existing in vivo at one stage of development may even be harmful to an embryo at a later stage of development.

Some of the disadvantages of the static-based Petri-dish culturing system may be circumvented by culturing the embryo in a culturing system capable of perfusing the embryo with a growth medium appropriate for its developmental stage. Such a system should be sized appropriately to match the size of the embryo and to more closely resemble the conditions existing in vivo. Furthermore, it is important to work in small scale to minimise the consumption of expensive growth media typically required by such mammalian cells.

Many so-called microfluidic devices have now been described for conducting various types of analysis or for culturing cells. These devices are often created using various principles, which are commonly inspired by the progress made in the 1970'ies with silicon-based technology for microelectronics. Examples of microfluidic applications are DNA-analyses involving principles such as the polymerase chain reaction for e.g. detection of single-nucleotide polymorphisms or assays for proteins using, e.g. capillary electrophoresis.

‘True’ microfluidic devices (e.g. with fluidic channels in the order of 100 μm diameter or less) do however suffer from a number of drawbacks, some of which are particularly pronounced for cell culturing devices designed for perfusion-type operation. As seen from the Hagen-Poiseuille equation the pressure drop in an e.g. 100 μm-channel with a flow becomes very large, putting high demands to a pump intended for operating at this scale, since such a pump must be able to precisely dispense very small volumes against a considerable back pressure. For this reason flows are often generated at this scale using so-called electroosmotic flow where a flow is created in a saline solution by exposing it to a large electrical potential. Such electroosmotic flow is however ill suited for systems involving live (mammalian) cells.

Another problem encountered in microfluidics is one related to the ‘connection to the outside world’. Most equipment employed in biological labs, such as pumps and analytical equipment, is so much larger than microfluidic equipment that integration between the two scales becomes problematic. Connection points for a tube as small as 250 μm-diameter (as is readily available) to a chip are difficult to handle for the lab worker, and moreover may quickly introduce dead volumes several times the size of the volume of the microfluidic system. This problem is especially important for perfusion-type cell culture devices where the operational complexity and the long residence times of fluids in tubes connected to a microfluidic system increase the risk of upstream contamination. In the case of culture of mammalian embryos the culture time can amount to five days or more.

For bioreactor systems working at small scales it is of course possible to switch between different growth media and conditions according to a predetermined sequence of events. However, in order to more fully optimise the growth conditions of the cells in a bioreactor the bioreactor may be equipped with appropriate sensors which communicate with a computer or similar capable of sending commands to actuators of the bioreactor. This way a feedback system may be created to respond to changes in the environment to e.g. maintain constant environmental conditions.

As discussed below some steps have been taken to approach the above problem.

WO07/044699 describes a microbioreactor fitted with integrated fluid injectors where the fluid injectors may comprise reservoirs for liquids. These bioreactors are useful for setting up parallel studies of especially microbial strains of commercially relevant production organisms within the area of metabolic engineering studies. Integrated sensors in the growth chambers of the bioreactors of WO07/044699 allow signals from the sensors to be used in a feedback control set-up for adjusting the environmental conditions, typically pH, within the growth chamber by injecting liquid from the reservoirs.

The reservoirs of the fluid injectors of the reactors of WO07/044699 are typically constructed from an elastomeric material and further comprise valves, so that the flow from the reservoirs may be controlled using pneumatic actuation.

In order to optimise the supply of gas to the growth chambers through permeable polydimethylsiloxane (PDMS) membranes the chambers of WO07/044699 are generally flat (i.e. one spatial dimension of the growth chamber is at least approximately 10 times smaller than the other two spatial dimensions of the growth chamber), with a PDMS membrane facing one of the two larger surfaces. Typical volumes of the growth chambers are given as from 5 to 500 μL. Mixing of the liquids in the growth chambers is also obtained using pneumatic actuation via the PDMS membrane.

The principles underlying the microbioreactors of WO07/044699 make them suited for performing fed-batch type fermentations of cells by pulsing liquids into the growth chamber to maintain constant environmental conditions. However, the pulsating supply of liquid and the limited size of the reservoirs (i.e. 15-25 μL) relative to the size of the growth chamber make the systems ill-suited for long term perfusion-like operation, such as chemostat culture or continuous supply of medium to cells in the chamber (i.e. with a reservoir size of 25 μL and a pulse size of ˜270 nL fewer than 100 pulses are available). This principle also seems badly suited for culturing cells with changing requirements during their growth cycle. Furthermore, while the flat design of the growth chambers allows an efficient oxygen supply to the cells this same design also necessitates vigorous agitation to mix the pulsed liquid. Such conditions may be damaging to cells more fragile than bacterial or fungal cells.

WO07/047826 describes a microfluidic cell culture device, which employs an oil overlay layer to prevent evaporation of liquid from a microfluidic chamber and to allow access to a growth chamber in the device. The device contains a funnel-shaped growth chamber and a reservoir connected via a microchannel in the bottom of a PDMS substrate comprising the chambers. With the aid of a membrane created from an elastomeric material and a so-called pin actuating device it is possible to create a peristaltic movement of liquid between the chambers. This peristaltic movement may be used to create a “back and forth-type of fluid supply, wherein the fluid level in the well increases and then decreases cyclically”. However, outside supplies may also be used to apply liquid to the growth chamber of the device of WO07/047826.

The devices of WO07/047826 may contain optical, electrical or electromechanical sensors to determine states or flow characteristics of elements of the microfluidic device. The document further mentions the option to employ multiple reservoirs for the supply of nutrients, growth factors, and the like using the active portions created from the combination of elastomeric membranes and pin actuating devices. However, the devices seem ill suited for conducting long-term perfusion type growth experiments, as there is a need to use an outside supply of fluid.

Petronis et al. (2006, BioTechniques 40:368-376) describe a microfluidic bioreactor device for long-term culture of HeLa cells. This device is constructed from thermoplastic polymeric materials and contains an integrated indium tin oxide film to heat the growth chamber. The chamber further contains a temperature sensor, and a set-up with a computer allows cooperation between sensor and heat element so that the temperature of the chamber may be controlled in a feed-back fashion. The small size of the bioreactor of Petronis et al. (2006) allows quick control of the temperature in the chamber. However, the device is simply a scaled-down version of a classic fermenter with liquids being supplied from external reservoirs using external pumps. Such a design with external liquid supplies makes possible a virtually unlimited culturing time, though the complexity of setting up an experiment makes the device best suited for use in an academic environment. Additionally, the feedback set-up is mainly suited for maintaining constant conditions and does not indicate how it may be employed to control the liquids applied to the growth chamber.

WO2005/123258 also describes a microfluidic bioreactor device employing sensors to control the environment of cells growing in a chamber in a feed-back mechanism. The bioreactor of WO2005/123258 utilises a premixing chamber located externally relative to the culture chamber. However, the bioreactor seems badly suited for conducting ‘traditional’ perfusion-type cell culturing as the premixing chamber and the growth chamber are connected in a circuit with the option to adjust the liquid composition (in the premixing chamber) with liquids supplied from an external reservoir.

Most microfluidic devices described to date are, however, analytical devices aimed at providing abstract data about cells or biological compounds in sample liquids in the devices. The aim of in vitro fertilisation procedures will in contrast be the embryo obtained in the culturing process. Therefore, a device designed for perfusing a cell, such as a fertilised oocyte, should provide easy access to the culturing chamber in order to allow the cell to be placed in the chamber, and especially also to be gently removed after the culturing period. This feature is not necessary in fluidic devices designed only for data acquisition where appropriate sensors may be integrated into the device allowing data to be extracted from the system without physically removing the cells.

WO2003/087292 describes an automated tissue engineering system. The system comprises inter alia a bioreactor supported by a housing, where the bioreactor may facilitate physiological cellular functions and/or the generation of a tissue construct. The system further comprises a fluid containment system comprising liquid reservoirs. The bioreactor may have a lid providing access to the bioreactor via a sampling port, and the bioreactor may have sensors for e.g. oxygen, temperature and pH and it may also be equipped with a camera. The device of WO2003/087292 is suited for such operations as digestion of tissue biopsy, cell sorting, cell washing, cell concentrating, cell seeding, cell proliferation, cell differentiation, cell storage, cell transport, tissue formation, implant formation, where the tissue may originate from bone and cartilage. Thus, this system is suited for cells, such as embryonic stem cells, adult stem cells, osteoblastic cells, pre-osteoblastic cells, chondrocytes, pre-chondrocytes, nucleus pulposus cells, skeletal cells, skeletal progenitor cells derived from bone, bone marrow or blood, including stem cells.

The scale of the bioreactor system of WO2003/087292 is not specified although a human cartilage biopsy of 100-500 mg is mentioned as relevant. It is further mentioned that 100 mg of biopsy tissue may give rise to approximately 200,000 to 500,000 cells in the system. Thus, the number of cells in the bioreactor suggests that this system is ill suited for in vitro fertilisation purposes where the aim is a cluster of cells derived from a single fertilised egg after a few divisional cycles.

There is moreover no indication of the exact nature of the “sampling port”. In particular, it is not described that the bioreactor may be accessed during perfusive operation.

The system of WO2003/087292 has been further developed as described in WO2005/116186. In WO2005/116186 it is claimed that for the differentiation bioreactor for implant formation, the design has been improved. However, WO2005/116186 does not detail how the bioreactor may be accessed via the lid, nor does it specify how the bioreactor may be accessed during perfusive operation.

Despite the efforts discussed above a system has yet to be described to solve the problems of integrating fluid supplies in a fluidic device intended for perfusion type operation on a scale and time appropriate for mammalian embryos. It is an aim of this invention to provide a mesoscale bioreactor platform, which is suited for culturing mammalian oocytes and embryos taking into account the different requirements to growth conditions during the development of the embryo as well as the period of time necessary for such culture.

### DISCLOSURE OF THE INVENTION

The present invention relates to a mesoscale bioreactor platform comprising two or more liquid reservoirs in fluid communication with a culture chamber, which chamber is in fluid communication with an exit, wherein the platform is provided with means allowing the chamber to be perfused with a flow of liquid from one or more of the liquid reservoirs.

The integrated reservoirs for liquids remove the need for external supplies of liquid to the culture chamber during cell culture experiments requiring perfusion of liquids, and furthermore minimises the risk of upstream contamination. Moreover, with two or more reservoir it is possible to supply the culture chamber with different liquids corresponding to the contents of each of the reservoirs, or the culture chamber may be supplied with mixtures from two or more reservoirs.

In certain embodiments the mesoscale bioreactor platform is constructed so that the culture chamber is accessible physically via a closable member or an elastic membrane. This construction allows manipulation of the contents of the culture chamber while maintaining a minimal risk of upstream contamination. The closable member of the mesoscale bioreactor platform may have the form of a lid, which may be hinged or sliding, and the elastic membrane may have a self-sealing capability.

In one embodiment the mesoscale bioreactor platform is constructed so that the means allowing the culture chamber to be perfused comprises a flexible region allowing the volume of the reservoir to be adjusted. The flexible region may be created from a flexible material in the form of a membrane. In another embodiment the means allowing the culture chamber to be perfused comprises an air inlet allowing an external connection to the reservoir which air inlet comprises a filter with a pore size of around 0.1 μm to around 0.5 μm. In either of these embodiments the application of a positive relative pressure to the outside of the membrane or the filter, or a negative relative pressure to the exit of the mesoscale bioreactor platform will create a flow of the liquids contained in the reservoirs towards the culture chamber.

The mesoscale bioreactor platform of the present invention may advantageously be used for culturing mammalian cells, and in this embodiment the culture chamber comprises one or more mammalian cells. The mesoscale bioreactor platform of the present invention is especially suited for use in in vitro fertilisation procedures. When employed for the culture of mammalian cells, the reservoirs comprise medium, biologically or biochemically active molecules and/or buffers.

The present invention further relates to a control unit for the mesoscale bioreactor platform, which control unit comprises means for controlling the flow rate of the liquid from the two or more reservoirs to the culture chambers. When the mesoscale bioreactor platform is connected to the control unit, the control unit can thus create a flow from the reservoirs to perfuse the culture chamber in the mesoscale bioreactor platform.

The means for controlling the flow rate may e.g. be capable of applying a positive relative pressure to said reservoirs and/or applying a negative relative pressure to said exit. The pressure may be created by a physical specimen, such as a piston, or air/gas pressure. It is preferred to use the pressure from a gas to provide for the flow. Application of a positive relative pressure to the liquid in the reservoirs may further allow the liquid to be saturated with the gas applied.

In another embodiment the control unit further comprises at least one sensor capable of measuring pH, dissolved oxygen (O₂), carbon dioxide (CO₂), glucose, nutrients, vitamins, metabolites, flow velocity, temperature, optical density, a fluorescent signal, specific proteins or enzymes, or DNA's or RNA's. The sensor may be located to measure the parameter value of the liquid in the culture chamber or downstream from the culture chamber.

In yet another embodiment the control unit further comprises a data processing unit capable of collecting signals from the sensor and using the information from the collected signals for controlling the flow rate from each of the two or more reservoirs. The presence of an optional mixing section may further ensure that liquids from two or more reservoirs are mixed. In this embodiment the signals recorded by the sensors may thus be employed to control the flow from the reservoirs to the culture chamber in a feed-back fashion. For example, the parameter values may be set to lie within a predetermined range, and should a signal from a sensor indicate that the parameter value may migrate outside this range, the flow from the reservoirs may be adjusted so as to bring the parameter value back within the predetermined range.

The invention also relates to a system comprising the mesoscale bioreactor platform and the control unit wherein the mesoscale bioreactor platform is comprised in a cartridge, which fits into the control unit. This system allows the mesocale bioreactor platform to be inserted quickly and easily into the control unit. The insertion will ensure that the reservoirs are connected with the pressure supply of the control unit.

### BRIEF DESCRIPTION OF THE FIGURES

FIG. 1 shows a schematic perspective drawing of a mesoscale bioreactor platform.

FIG. 2 shows a schematic drawing of a culture chamber of a mesoscale bioreactor platform.

FIG. 3 shows a top view of a mesoscale bioreactor platform showing elements of specific embodiments the platform.

FIG. 4 shows a control unit for a mesoscale bioreactor platform.

FIG. 5a is a diagram of an algorithm for feedback regulation of temperature.

FIG. 5b is a plot of temperature vs. time using the algorithm to change temperatures.

### DETAILED DESCRIPTION OF THE INVENTION

The present invention relates to a mesoscale bioreactor platform, a control unit for the bioreactor platform, and a system comprising the platform and the control unit.

The term “bioreactor” of the present invention covers systems and devices suited for culturing biological cells. The disclosed bioreactors are especially suited for mammalian cells. In a preferred embodiment the mammalian cells are cells related to in vitro fertilisation, and the cells will comprise spermatozoa, oocytes, and/or embryos. However, as will be obvious to those skilled in the art the bioreactor platform may also be useful for other mammalian cell types, such as stem cells or cells of the immune system, such as monocytes, dendritic cells, T-cells and the like. In a preferred embodiment the mammalian cells are human cells. Furthermore, a mesoscale bioreactor platform as disclosed in the present invention may also be of utility in the culturing of cell types other than mammalian cells. For example, bacterial, yeast, fungal, plant, or insect cells may also be cultured in the bioreactor platform disclosed herein.

In the context of this invention the term “mesoscale” is intended to cover a range of sizes where the smallest dimension of channels is in the range from around 100 μm to around 3 mm, although the channels may also contain constrictions. Likewise the culture chamber may be of a depth of around 500 μm to around 5 mm or more, and the largest horizontal dimension may be from around 1 mm to around 50 mm. The size of the reservoirs must be sufficient to culture cells under perfusion conditions. It can generally be said that fluids in mesoscale fluidic systems will be flowing under laminar conditions, and fluidic systems with channels or chambers different from those defined above may well be described as “mesoscale” as long as fluids contained in the systems flow under laminar conditions.

In a certain embodiment the largest horizontal dimension of the culture chamber is in the range from around 2to around 6 mm. In another embodiment the largest horizontal dimension of the culture chambers is in the range from around 20 to around 30 mm. Within the range of flow-rates typically employed in the mesoscale bioreactors of the invention the liquids will be moving in an essentially laminar flow.

The bioreactor platform of the present invention is suited for operation under perfusion conditions. In this context the terms “perfusion” or “perfuse” mean that a generally continuous flow is applied to the culture chamber(s) of the device. This continuous flow is not limited to a certain flow-rate, but during the course of an experiment with a bioreactor platform of the present invention several different flow-rates may be employed. Suitable flow-rates are from around 100 nL/min to around 200 μL/min or more. It should be emphasised that the flow may also be stopped if necessary, e.g. for performing various operations involving the contents of the culture chamber(s). Furthermore, intermediate operation allowing rest to the biological cells is also contemplated.

In some embodiments the culture chamber is constructed in a way to allow physical access to the chamber. In this context the term “physical access” means that a tool may be inserted into the liquid in the culture chamber to manipulate the contents of the culture chamber. This manipulation may be to insert or remove one or more cells from the culture chamber, or it may involve manipulations of cells already present in the culture chamber.

In one embodiment, the control unit of the present invention contains a “data processing unit”. With this term is meant a computer or similar device, which is capable of collecting signals from the sensors in the control unit and converting them to data understandable to an operator. The data processing unit is also capable of sending commands to equipment controlling environmental parameters experienced by cells in the culture chamber.

The data processing unit may further be set up so that a “temporal log” can be created for the mesoscale bioreactor platform. With the term “temporal log” is meant that signals generated by the sensors of the control unit or other data are collected by the data processing unit together with the time at which the signals were collected. Thus, for each mesoscale bioreactor platform it will be possible to know the conditions that existed for any cells in the culture chamber at a given point in time during the culturing period. This information will similarly be available already before the end of the culturing period, so that e.g. decisions may be taken based on knowledge of the data in the temporal log.

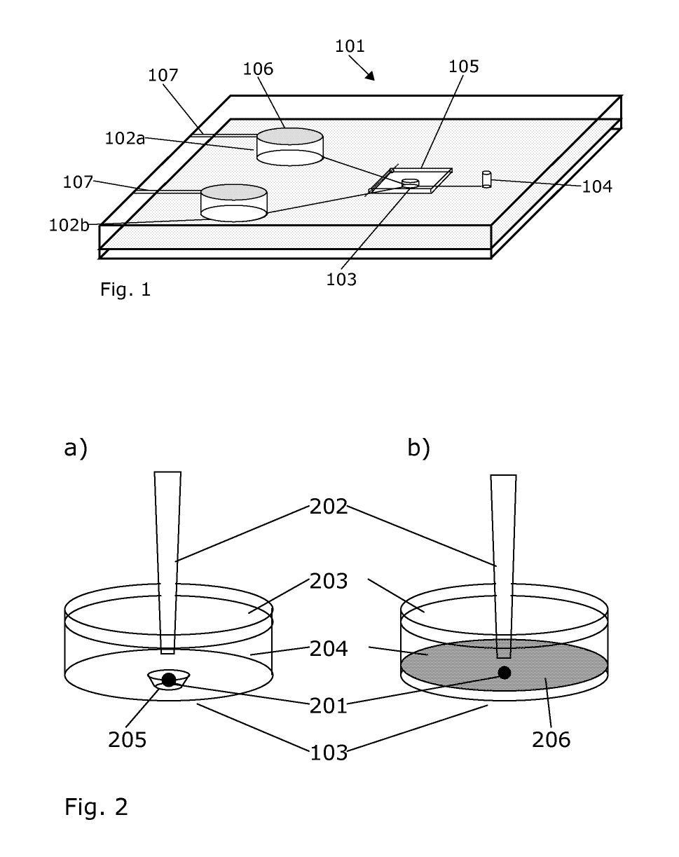

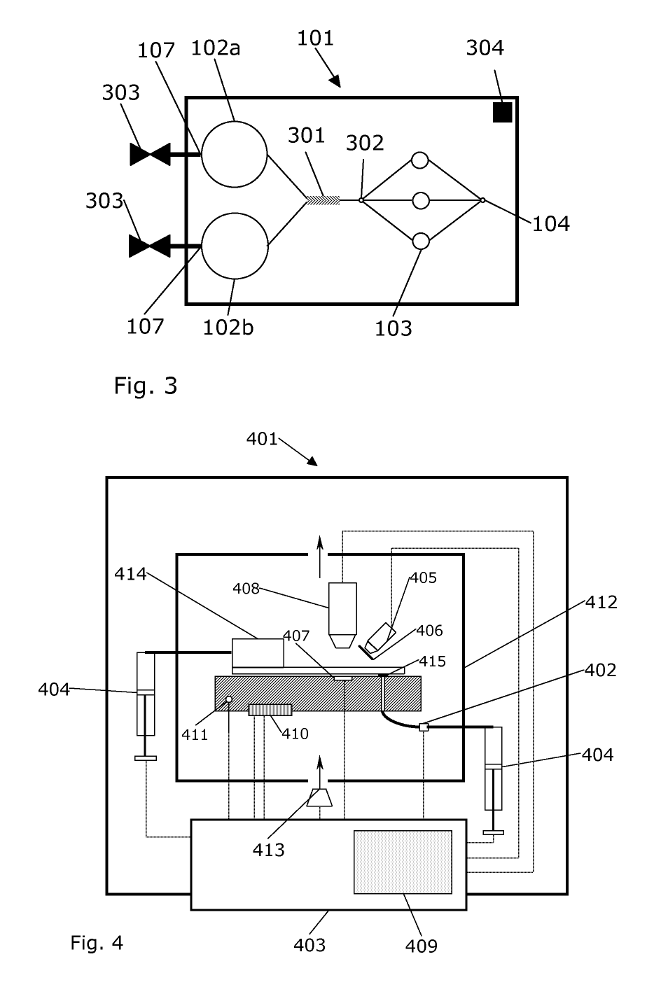

The mesoscale bioreactor platform 101 of the present invention is particularly suited for use in in vitro fertilisation procedures. More specifically the mesoscale bioreactor platform 101 is suited for culturing embryos or oocytes 201 following insemination of an oocyte 201 with spermatozoon. For this purpose a fertilised oocyte 201 is positioned in the culture chamber 103 with the aid of a tool 202, such as a pipette or hypodermic needle. In a preferred embodiment the culture chamber 103 of the mesoscale bioreactor platform 101 is designed so as to allow physical access to the culture chamber. The mesoscale bioreactor platform 101 may also be used for fertilising an unfertilised oocyte. For this purpose the reservoirs 102 may comprise purified or unpurified spermatozoa, or hyaluronidase for cumulus removal. The mesoscale bioreactor platform 101 is illustrated schematically in FIG. 1, and the culture chamber 103 of the platform in FIG. 2. Additional features of specific embodiments of the bioreactor platform 101 are illustrated in FIG. 3.

In one embodiment, the culture chamber 103 may thus comprise an elastic membrane of a material with a self-sealing capability. Thus, when the membrane is penetrated with a tool 202, such as a needle, a hypodermic needle, a lancet or a canula, the tool 202 will gain access to the fluids contained in the culture chamber 103 comprising the membrane. Upon removal of the tool 202 the self-sealing capability will ensure that the chamber is sealed and undesired leaks are prevented. Therefore, the fertilised oocyte 201 may be positioned in the culture chamber 103 by placing it in e.g. a hypodermic needle with the aid of a syringe, penetrating the elastic membrane with the tip of the hypodermic needle, and injecting the oocyte 201 in an appropriate position in the culture chamber. The culture chamber 103 may then be perfused with appropriate growth media from one or more of the reservoirs 102.

In another embodiment the culture chamber 103 comprises a closable member 105 to allow access to the chamber. The closable member 105 is designed so that in one setting it serves to enclose the liquids in the culture chamber 103 and prevent leaks when the culture chamber 103 is perfused with liquids, and in another setting the closable member 105 will allow physical access to the chamber using tools 202, such as a pipette, a needle, a hypodermic needle, a lancet or a canula or other tools to manipulate the contents of the culture chamber. The closable member 105 may be part of the mesoscale bioreactor platform 101 or it may be part of the control unit. In a preferred embodiment the closable member 105 is transparent.

The closable member 105 may advantageously take the form of a lid, which may be hinged or sliding. When the closable member 105 is in the form of a lid it is typically constructed from a rigid material, such as one or more thermoplastic polymer, though it may further comprise an elastomeric material, such as PDMS or a rubber. In this embodiment opening of the lid will allow e.g. the insertion of a pipette tip containing a fertilised oocyte to position the oocyte 201 appropriately in the culture chamber 103 before closing the lid and perfusing the culture chamber 103 with appropriate growth media from one or more of the reservoirs.

In another embodiment the liquids in the culture chamber 103 of the mesoscale bioreactor platform 101 comprise an aqueous liquid 204 and another liquid 203, which is essentially immiscible with the aqueous liquid 204. This essentially immiscible liquid 203 may be an oil of lower density than the aqueous liquid 204, such as paraffin oil, and it may be present as an overlay layer 203 on top of the aqueous liquid 204. Cells, such as oocyte 201, contained in the culture chambers will typically be present in the lower layer of the aqueous liquid 204. Such an overlay oil layer 203 may serve to minimise the perfused volume of aqueous liquid 204 in a culture chamber, prevent evaporation from a growth medium in the aqueous phase, and minimise biological contamination of the aqueous liquid 204. Specifically, the application of an oil layer may help maintain pH by preventing evaporation of CO₂. An oil layer may also help to maintain the correct temperature of the culture chamber. Application of an overlay oil layer 203 is useful when the mesoscale bioreactor platform 101 is fitted with an elastic, self-sealing membrane or a lid.

The culture chamber 103 of the mesoscale bioreactor platform 101 of the present invention is not limited to a particular shape. However, in a preferred embodiment the shape of the culture chamber 103 may be generally described as cylindrical with an essentially round circumference. The diameter of this circumference may be larger or smaller than the height of the cylinder. The height of the cylinder will normally follow the vertical axis. In one embodiment the diameter of the cylindrical culture chamber 103 may be from around 2 to around 6 mm, and in another embodiment it may be from around 20 to around 30 mm. The depth of these cylindrical culture chambers may be from around 0.5 to around 2 mm. In other embodiments the culture chamber 103 may be generally box-shaped. This box-shape may take the form of a generally flat box with rectangular sides, or the box may be closer to a cube in shape. In one embodiment the culture chamber 103 may be of a width of around 5 to around 10 mm with a length of up to around 50 mm. The depth of such box-shaped culture chambers may be from around 0.5 to around 2 mm.

The culture chamber 103 of the present invention may also be fitted with one or more depressions 205 in the bottom surface of the chamber. These depressions 205 may be generally cylindrical with horizontal and vertical dimensions of similar sizes. The dimensions are typically around 500 μm. The depression 205 is suited for retaining the one or more mammalian cells 201. It is particularly suited for retaining a fertilised oocyte 201. Thus, prior to the culture of an embryo a fertilised or unfertilised oocyte 201 may be placed, with e.g. a pipette or a hypodermic needle, in the depression in the culture chamber 103. If only one depression is present it may be generally centrally located in the bottom surface of the culture chamber. If more than one depression are present these may be located along a line in the bottom surface, or they may be laid out in a suitable pattern, such as that determined by the intersections in a mesh of rectangular, triangular or hexagonal cells, or a mesh similar to a spider's web, or along the perimeters of concentric circles.

The mesoscale bioreactor platform 101 of the present invention is not limited to a single culture chamber. Actually, in some embodiments the bioreactor platform 101 comprises several culture chambers, for example 10-20 culture chambers. These culture chambers may be arranged in one or more groups of serially connected culture chambers. Each group may be connected in parallel with said reservoirs. Thus, the platform may contain a single culture chamber, multiple culture chambers connected in a single series, multiple culture chambers connected in parallel, or groups of serially connected culture chambers where each group is connected in parallel.

In a preferred embodiment the mesoscale bioreactor platform 101 of the present invention contains 10 cylindrical culture chambers of around 4 mm diameter and around 1.5 mm depth each with a single depression located in the bottom surface, and the volumes of each of the culture chambers is around 20 μL or less. In this embodiment the culture chambers are connected serially with the reservoirs.

In another preferred embodiment the mesoscale bioreactor platform 101 of the present invention contains one cylindrical culture chamber 103 of approximately 20-40 mm diameter. In this embodiment the culture chamber 103 has 8-20 depressions.

The inner surface of the culture chamber may be smooth or rough, although for certain applications the culture chamber may be fitted with a scaffold 206 supporting cellular growth. Such a scaffold 206 may be part of the material making up the culture chamber, or it may be provided in the form of an insertable imprint. The scaffold 206 may be shaped so as to resemble a biologically occuring interface, and it may involve a physically imprinted pattern, or a pattern with a varied pattern of hydrophilic and hydrophobic sites, or a combination of the two. In yet another embodiment the scaffold 206 may be functionalised chemically with species appropriate to cell binding, such as proteins, charged groups, cells, cells debris or the like.

The fluidic structures of the mesoscale bioreactor platform 101 may also further comprise a mixing section 301 which will generally be located between the reservoirs 102 and the culture chamber 103. Thus, the fluid streams from the two or more reservoirs 102 may therefore be mixed before the fluid reaches the culture chamber. Such a mixing section 301 may comprise internal structures on an inner surface of a channel section, such as a herring-bone structure or a chaotic mixer, or it may comprise a length contributing element, such as a meander channel or a spiralling channel, allowing the liquids to be mixed by diffusion. The platform may also comprise a manifold 302 or similar structure dividing the flow from the reservoirs 102 into a number of channels.

The reservoirs 102 of the mesoscale bioreactor platform 101 of the present invention are generally of larger volumes than the culture chamber. In a preferred embodiment the volumes of the reservoirs 102 are at least 10 times larger than the volume of the chamber. In another preferred embodiment the volumes of the reservoirs 102 are at least 20 times larger than the volume of the chamber. It is an aspect of the invention that a flow from the reservoirs 102 may be generated by applying a positive relative pressure to the reservoirs 102 or by applying a negative relative pressure to the exit 104 of the mesoscale bioreactor platform 101 which is in fluid communication with the reservoirs 102 via the culture chamber.

In one embodiment the part of the structure enclosing the reservoirs 102 comprises a flexible region 106 allowing the volume of the reservoir to be adjusted. In this embodiment applying positive relative pressure to the outside surface of the flexible material will create a flow of the liquid contained in the reservoir towards the culture chamber 103 of the mesoscale bioreactor platform. The flexible region 106 preferentially comprises a polymeric material, such as PDMS, rubber, polyethylene or the like. The material may be attached to the substrate containing the reservoirs 102 using methods well-known within the art, such as adhering by glue, ultrasonic welding, laser welding, welding using heat, clamping, stitching or the like.

In a preferred embodiment the reservoirs 102 comprise an air inlet 107 allowing an external connection to the reservoir. This air inlet 107 preferentially comprises a filter with a pore size of around 0.1 μm to around 0.5 μm, so that e.g. particulate material may be prevented from entering the reservoir. The air inlet 107 may be connected to a gas supply allowing the application of a positive relative pressure to force the liquids out of the reservoir and into the culture chamber. The air inlet 107 also affords that a negative relative pressure applied to the exit 104 of the mesoscale bioreactor platform 101 will draw the liquid of the reservoir into the culture chamber. Positive and negative relative pressures applied to the reservoirs 102 or the exit 104, respectively, may further be used to control the liquid volume in the culture chamber 103.

In these embodiments it will also be possible to control the distribution of liquids from different reservoirs 102a,b, respectively, by using valves 303 to control the access of air or gases via the air inlet 107 to the reservoir 102. For example, by closing a valve 303 connected to one reservoir 102 it will be ensured that the flow of liquid to the culture chamber 103 will stem from other reservoirs 102. Such valves 303 will be located externally relative to the mesoscale bioreactor platform. The valves 303 may be applied for mesoscale bioreactor platforms intended for operation by application of negative relative pressure to the exit 104 of the platform 101 as well as for platforms intended for operation by application of positive relative pressure to the reservoirs.

The reservoirs 102 of the mesoscale bioreactor platform 101 may take the form of generally flat cylinders or they make take the form of long channels. Such channels may be arranged in a meander structure. When the reservoirs 102 are cylinder shaped the air inlet 107 will generally be located near or at the top of the cylinder, and the channel connected to the culture chamber 103 will generally be located near or at the bottom of the cylinder. The bottom of the cylinder may have a flat surface, or the surface may be conical or funnel-shaped, it may be sloped or of a more complex shape combining the above characteristics.

In one embodiment the mesoscale bioreactor platform 101 is further fitted with a radio frequency identification (RFID)-tag 304. This RFID-tag may allow a quick and convenient identification of the bioreactor platform. Identification of the bioreactor platform 101 is advantageous when the information contained in the RFID-tag is linked to the identity of a person providing the cells being cultured in a given bioreactor platform.

The mesoscale bioreactor platform of the present invention is preferably constructed from essentially transparent materials with hydrophilic surfaces, although well-defined regions of hydrophobic surfaces may also be used. The construction material is preferably one or more thermoplastic polymers, although other materials, such as glass, silicon, metal, elastomeric polymers, may also be used.

Channels and chambers of the mesoscale bioreactor platform of the present invention may be formed by joining a first substrate comprising structures corresponding to the channels and chambers with a second substrate. Thus, the channels and chambers are formed between the two substrates upon joining the substrates in layers. The mesoscale bioreactor platforms are not limited to two substrate layers. In certain embodiments multiple substrates may be used where each of the substrates may comprise structures for channels and chambers as appropriate. These multiple substrates are then joined in layers so as to be assembled as a mesoscale bioreactor platform.

The structures corresponding to the channels and chambers in the substrates may be created using any appropriate method. In a preferred embodiment the substrate materials are thermoplastic polymers, and the appropriate methods comprise milling, micromilling, drilling, cutting, laser ablation, hot embossing, injection moulding and microinjection moulding. These and other techniques are well known within the art. The channels may also be created in other substrate materials using appropriate methods, such as casting, moulding, soft lithography etc.

The substrate materials may be joined using any appropriate method. In a preferred embodiment the substrate materials are thermoplastic polymers, and appropriate joining methods comprise gluing, solvent bonding, clamping, ultrasonic welding, and laser welding.

The present invention also relates to a control unit 401 for the mesoscale bioreactor platform 101. This control unit 401 is capable of applying positive relative pressure to the reservoirs 102 of the bioreactor platform and/or a negative relative pressure to the exit 104 of the bioreactor platform. The application of such pressures will create a flow of liquid from one or more of the reservoirs towards the culture chamber 103 of the bioreactor platform. The gas applied to the reservoirs 102 with a positive relative pressure or aspired into the reservoirs 102 with a negative relative pressure applied to the exit 104 may be of any composition. Thus, the gas may be air or it may be premixed with e.g. 2-10% CO₂ and/or it may be a trigas with 2-20% O₂. A control unit 401 according to one embodiment of the present invention is illustrated schematically in FIG. 4.

In one embodiment the control unit 401 comprises a liquid pump 404 in fluid communication with the exit of the bioreactor platform via an appropriate conduit, so that the pump 404 may be used to aspire liquids from the bioreactor platform via the exit 104 and thereby generating a flow from the reservoir(s) to the culture chamber. This pump 404 may be a peristaltic pump, a piston pump, a syringe pump, a membrane pump, a diaphragm pump, a gear pump, a microannular gear pump, or any other appropriate type of pump.

In another embodiment the control unit 401 comprises one or more pumps 404 suited for pumping gases. In this embodiment the one or more pumps 404 will be in fluid communication with the air inlets of the bioreactor platform. The control unit 401 may comprise a pump 404 for each of the reservoirs of the bioreactor platform or it may comprise a single pump 404, or the number of pumps 404 may fall between these two values. In case the control unit 401 has fewer pumps 404 than the number of reservoirs the control unit 401 will also comprise appropriate valves enabling the composition of liquids supplied to the growth chamber of the mesoscale bioreactor platform to be controlled with respect to the contents of the reservoirs. Pumps 404 employed in this embodiment may be a piston pump, a syringe pump, a membrane pump, a diaphragm pump, or any other appropriate type of pump.

In yet another embodiment the control unit 401 comprises both one or more pumps 404 suited for pumping gases to the air inlets of the bioreactor platform and a liquid pump 404 in fluid communication with the exit of the bioreactor platform.

The control unit 401 of the present invention preferentially comprises one or more sensors 402 set up to measure an environmental parameter of a liquid in the mesoscale bioreactor. These parameters are typically pH, dissolved oxygen (O₂), carbon dioxide (CO₂), glucose, nutrients, vitamins, metabolites, flow velocity, temperature, optical density, a fluorescent signal, specific proteins or enzymes, or RNA's or DNA's, although other parameters may also be appropriate. These sensors 402 may be integrated into a conduit connected to the exit of the mesoscale bioreactor in order to perform measurements of the liquids downstream from the culture chamber. In another embodiment the sensors are set up to perform measurements of the liquids in the culture chamber.

In addition to these sensors 402 the control unit 401 may be equipped with optical detection and observation systems. These optical systems could comprise a light source 405, such as a light emitting diode (LED), a light bulb, a mercury lamp or the like, appropriate filters 406 and a photodetector 407. LED's may be of a type to emit white light or they may be of a type emitting light of a relatively narrow range of wavelengths. This latter type of LED's are appropriate for measuring optical densities of wavelengths corresponding to that characteristic for the LED or for exciting fluorescent entities to emit light of a characteristic wavelength, which may then be detected as a fluorescent signal. Alternatively mercury lamps are also appropriate components for detection of fluorescent signals when coupled with suitable light filters 406 and photodetectors 407.

In a preferred embodiment the control unit 401 is equipped with a digital or an optical microscope 408. In this embodiment the control unit 401 may also be equipped with one or more light sources 405. In another embodiment the control unit 401 is further equipped with a display 409 allowing the culture chamber and its contents to be monitored via the microscope 408. In yet another embodiment the control unit 401 further comprises visualisation software capable of monitoring any cells growing in the culture chamber and, depending on the morphology of the cells, sending commands to equipment controlling the pressure applied to the reservoirs and/or the exit of the mesoscale bioreactor platform, a gas supply 413 generating a laminar air flow and a heating/cooling system 410 to regulate the temperature of the mesoscale bioreactor platform. The morphology of the cells may involve the number, sizes, shapes or orientation of cells or a combination. The morphology may also involve fluorescent signals or colorimetric signals from the optical detection systems.

The control unit 401 may also be fitted with a system 410 to regulate the temperature of the mesoscale bioreactor platform. In a preferred embodiment this system 410 further comprises one or more temperature sensors 411 coupled with a data processing unit 403 allowing the temperature to be controlled via the data processing unit 403. The temperature regulation system 410 may for example comprise an aluminium block shaped to house the bioreactor platform and comprising a coil of an electrically conductive wire, a peltier element, tubes for a heating and/or cooling liquid, or similar. In a preferred embodiment the control unit 401 is equipped with an aluminium block with a heating element 410 and a temperature sensor 411; this temperature sensor 411 is connected to the data processing unit 403. In another preferred embodiment the control unit 401 is equipped with a block of a transparent material, such as glass, containing the heating element 410. The data processing unit 403 in this embodiment may use signals from the temperature sensor 411 in a so-called model predictive control (MPC) algorithm to precisely regulate the temperature of the mesoscale bioreactor platform by controlling the power supplied to the heating element.

The control unit 401 comprising the mesoscale bioreactor platform may advantageously contain a compartment 412 surrounding the mesoscale bioreactor platform, which compartment 412 may be supplied with gases to create a laminar air flow (LAF) 413 around the bioreactor platform. This “laminar air flow” 413 describes a situation where air is flowing through the compartment 412 in a pattern virtually free of turbulence, and these conditions may serve to lift air-borne particular material away from the culture chambers thereby preventing contamination of the cells in the chamber, e.g. when the culture chamber is open for physical access. In one embodiment the laminar air flow 413 is supplied to the compartment 412 from below the bioreactor platform to one or more exits above the bioreactor platform so that the air is moving in a generally upwards direction. In another embodiment, the laminar air flow is oriented along the surface of the bioreactor platform, i.e. in a substantially horizontal orientation. The laminar air flow 413 may be composed of atmospheric air, though in a preferred embodiment the content of CO₂ is increased relative to atmospheric air to e.g. approximately 2-10%, or more preferably 5%. In other embodiments the content of O₂ may also be increased or decreased. The pressure of the laminar air flow may be essentially identical to that of the ambient air. However, the pressure is preferably increased relative to the ambient air. When the content of CO₂ or O₂ or other gases of the laminar air flow is increased, the flow may further be controlled so that the pH of liquids in the bioreactor platform may be regulated. The linear flow velocity of the laminar air flow is typically in the range 50 μm/s to 0.1 m/s.

In one embodiment the control unit 401 further comprises a data processing unit 403. This data processing unit 403 is capable of collecting signals from sensors 402 and/or 411 of the control unit 401 and sending commands to control the positive relative pressure applied to the reservoirs and/or to control the negative relative pressure applied to the exit of the bioreactor platform in order to control the flow-rates of liquids in the bioreactor platform 101, as well as sending commands to regulate the temperature or the laminar air flow as appropriate.

Control of these operational parameters may be based on a predetermined chronological series of events, or the commands may be based on the signals collected from the sensors 402 and/or 411 in the control unit 401, e.g. in a feedback-type loop. In case a predetermined sequence of commands is employed this could for example involve perfusing an embryo with growth medium from reservoir 102a for a set number of days at a given flow-rate before changing the perfusion to growth medium from reservoir 102b at the same or a different flow-rate for the remainder of the culturing period while at all times maintaining the temperature at 37° C.

An example of a set-up employing signals from the sensors 402 and/or 411 to determine the operational parameters could be that if the temperature sensor 411 indicates that the temperature is outside a set interval the data processing unit 403 will send a command to heat or cool the mesoscale bioreactor platform so that the temperature will once again be brought within the set interval. Likewise an indication from a pH sensor 402 that the pH is moving away from a set range the gas supply 413 may for example be adjusted so that an increased amount of CO₂ is applied to the compartment 412 containing the mesoscale bioreactor platform. Levels of O₂, glucose and other metabolites (pyruvate and lactate) and energy (ATP/ADP) may also be used for controlling the operational parameters.

The control of the operational parameters may also involve a more complex set of instructions employing signals from the sensors 402 and/or 411. For example, when a signal from a sensor 402 or 411 or an observation indicates that an event has occurred in the culture chamber the instruction set may contain an instruction for the data processing unit 403 to counter the event and maintain a stable parameter value for parameters such as pH, temperature, or nutrients perfused to the culture chamber, or the instruction set may contain an instruction for the data processing unit 403 to apply a new set of conditions to the cells in the culture chamber. This could for example be that when a certain morphology is observed for an embryo in the culture chamber, the flow is changed from the growth medium contained in one reservoir to that of the other reservoir. Thus, these conditions could involve parameters such as flow-rate, temperature, pH, distribution of flows from the different reservoirs, or the like.

The data processing unit 403 may also comprise a user interface allowing the operator to manually control the operational parameters (flow-rates in the channels and chambers, distribution of flow from different reservoirs, temperature, pH etc.). And thus, the control unit 401 may be set up to control the parameter values of the mesoscale bioreactor platform in a fully automated, pre-determined sequence of events, or a fully automated sequence of events determined by signals from the sensors 402 of the control unit 401, a manually operated sequence of events, or any combination of these operating principles.

Regardless of the principle of operation the data processing unit 403 may create a temporal log of the signals collected from the sensors of the control unit 401. This temporal log may also contain information about events in e.g. the culture chamber of a mesoscale bioreactor platform or commands employed to control environmental parameters for the platform. The temporal log may advantageously be coupled with the information from a RFID tag on the mesoscale bioreactor platform, and in one embodiment the control unit 401 comprises a sensor to read the RFID-tag. This way a temporal log may be easily linked with a data tag containing information about the origin of the cells in the bioreactor platform, such as the name and identity of the person providing the cells, as well as the identity of the operator.

The control unit of the present invention may simply be designed to hold a single mesoscale bioreactor platform. However, in another embodiment the control unit may contain e.g. up to six mesoscale bioreactor platforms in one control unit.

The present invention further relates to a system comprising the mesoscale bioreactor platform 101 and the control unit 401. In this system, the bioreactor platform 101 is preferably designed in the form of a cartridge 414 fitting in the control unit 401 of the invention. Insertion of the cartridge 414 into an appropriately designed seat or similar in the control unit 401 will ensure that the exit of the bioreactor platform is connected to the corresponding conduit of the control unit, and that the air inlets of the reservoirs of the platform are connected to the supply of positive relative pressure as appropriate.

Insertion of the cartridge 414 containing the bioreactor platform into its seat in the control unit will further allow the sensor(s) of the control unit to monitor liquids from the bioreactor platform, and it will also ensure efficient heat transfer between the temperature regulating system of the control unit and the bioreactor platform. When the cartridge 414 is inserted in the control unit any optical detection or monitoring systems being part of the control unit will be appropriately aligned with culture chambers or channels in the mesoscale bioreactor platform.

Thus, the application of a mesoscale bioreactor platform contained in a cartridge 414 fitting into an suitably designed control unit will allow a quick coupling between the mesoscale bioreactor platform and the control unit which will simplify the operation of the integrated system. In an example of a system comprising the bioreactor platform and the control unit, the control unit could contain an aluminium or a glass block with an appropriate temperature regulating system, such as a peltier element or a heating coil, and an integrated conduit and O-rings 415 ensuring efficient sealing upon insertion of the cartridge 414. The physical form of the aluminium or glass block will be such that there is only one possible way to insert the cartridge 414 so that the exit will be connected to the conduit of the control unit, and the air inlets of the reservoirs will be connected appropriately. The mesoscale bioreactor platform 101 and the matching control unit 401 may be designed such that the exit 104 is facing upwards or downwards by locating it on an upper or lower surface of the mesoscale bioreactor platform. Correct insertion of the cartridge 414 will preferably be obvious to the operator.

### EXAMPLES

### Example 1

### Construction of a Mesoscale Bioreactor Platform

A prototype mesoscale bioreactor platform consisting of four layers of substrate materials was designed using the 2D drawing software Auto-CAD LT (Autodesk, San Rafael, Calif., USA). The bioreactor platform design contained two cylindrical reservoirs of 16 mm diameter and 5 mm depth (1 mL volume), which were connected to a junction by two channels. Each reservoir had a channel allowing to connect the reservoir to the ambient surroundings. A channel from the junction led to three serially connected culture chambers of 4 mm diameter and 1.5 mm depth (similar to a volume of 20 μL). Each culture chamber had a depression of approximately 500 μm diameter and 200 μm in depth in the bottom surface. A waste channel led from the third culture chamber to the ambient surroundings.

The bottom layer of the design of the bioreactor platform was a rectangular plate (5 cm×8 cm size) with a through-hole of 500 μm diameter and the three depressions of the culture chambers. This plate was designed to be joined with a second substrate plate (of 4.5 cm×7.5 cm size) containing three through-holes corresponding to the culture chambers (4 mm diameter) and two additional through-holes (of 500 μm diameter) the positions of which matching the positions of the reservoirs in the above layer. The two 500 μm diameter holes were each connected with a channel (500 μm width) meeting in the junction to form a channel (500 μm width) leading from the junction to the first hole corresponding to a culture chamber; further channels connected the other culture chamber holes, and a final channel was designed to match the throughhole in the bottom layer (thus constituting a waste channel). All channels were designed to be created in the bottom surface of the second layer. A third layer (of 4.5 cm×2.5 cm size) merely contained two 16 mm diameter through-holes corresponding to the reservoirs. The fourth layer (of 4.5 cm×2.5 cm size) contained to channels (500 μm width) leading from positions corresponding to the centres of the two reservoirs to one edge of the plate. The channels of the fourth layer were designed to be created in the bottom surface of this plate.

The AutoCAD LT-designs were used to ablate the structures into substrates of poly(methyl methacrylate) (PMMA) using a Synrad Fenix Marker CO₂-laser (Synrad Inc., Mukilteo, Wash., USA). The transparent PMMA substrates were supplied by Rōhm GmbH & Co. (Plexiglas XT20070, Rōhm GmbH & Co., Darmstadt, Germany); the layer containing the reservoirs was of 5 mm thickness, all others were of 1.5 mm thickness. Prior to the ablation the AutoCAD LT-designs were converted to encapsulated post-script files and imported into the WinMark Pro software controlling the Synrad Fenix Marker CO₂-laser. Ablation was performed using laser settings, which will be well-known to those skilled in the art.

Following an appropriate annealing procedure at 80° C. to prevent stress cracking of the PMMA substrates, the bottom surfaces of the three uppermost substrates were dyed with an IR-absorber dye (Clear-Weld LD130, Gentex Corp., Carbondale, Pa., USA). The different layers were then welded together using a Fisba FLS Iron laser scanner (Fisba Optik AG, St. Gallen, Switzerland) capable of yielding a powerful ˜800 nm laser light. Initially the second substrate layer was welded to the bottom substrate layer, and subsequently the third and fourth layers were welded to the growing stack of layers. During the welding the substrates were pressurised appropriately using a vice created with glass that is transparent to the laser light. Optimal laser settings for efficient welding are well known within the art.

The three culture chambers of this prototype mesoscale bioreactor platform are open and accessible. During perfusion operation the culture chambers may be closed with e.g. a slab of PDMS.

### Example 2

### Construction of a Mesoscale Bioreactor Platform

A mesoscale bioreactor platform was designed and constructed as described in Example 1 except the three serially connected culture chambers (in the second substrate layer) were replaced with a single culture chamber of 20 mm diameter (volume 0.5 mL). The bottom of this single culture chamber contained six depression (approximately 500 μm diameter and 200 μm depth) placed on the perimeter of a circle of 10 mm diameter located in the centre of the chamber.

### Example 3

### Construction of a Control Unit

A suitable box of a polymeric material was selected to construct a prototype control unit for housing a mesoscale bioreactor platform. The size of the box was approximately 16×24×12 cm³. The box was fitted with a compartment consisting of a smaller box for containing an aluminium block (approximately 10×7×2 cm³), to function as a heat regulating element, and either of the mesoscale bioreactor platforms described in Example 1 or Example 2. The aluminium block was machined to exactly house the bioreactor platform, and a hole (1 mm diameter) was drilled in it in a location corresponding to the location of the exit of the mesoscale bioreactor platform. The opening of the hole was expanded to house a rubber O-ring (1 mm ID), and the exit hole fitted with a piece of 0.5 mm ID Teflon tube which was connected to micro-scale pH-electrode further being connected to a 2 mL syringe pump. The pH-electrode was connected to a sensor board, which was further connected to a PC running LabView (ver. 8, National Instruments, Austin, Tex., USA).

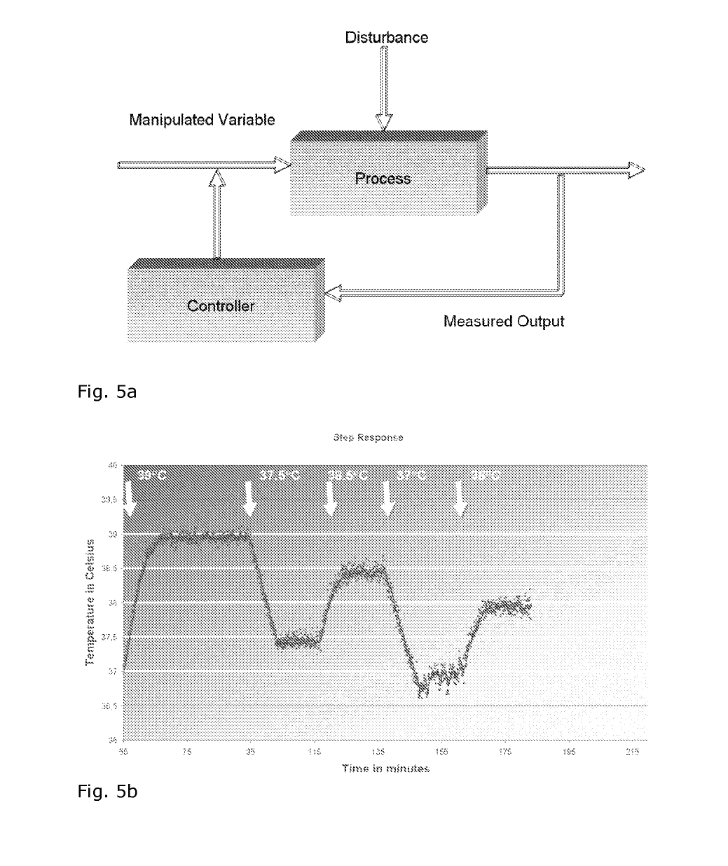

The aluminium block was further machined to house a temperature regulating element, either a peltier element or a heating coil, which was connected to a DC power supply. An electronic temperature sensor was integrated into the aluminium block. The electronic control for the heating element and the temperature sensor were both connected to the sensor board. A custom made LabView application was created to implement a model predictive control (MPC) algorithm for controlling the temperature on the basis of input from the temperature sensor. The principle of the algorithm is illustrated in FIG. 5a, and a test of the algorithm in FIG. 5b, showing the temperature measured for the aluminium block as a function of time following programmed changes in the temperature.

The compartment containing the aluminium block consisted of a transparent plastic box with a closable lid. The bottom side of this box had an approximately 1 cm diameter round hole which was connected to an air supply system capable of supplying the compartment with a laminar air flow surrounding the aluminium block.

The prototype control unit box was further equipped with two syringe pumps each fitted with a piece of (0.5 mm ID) tube allowing connection to the air inlets of the bioreactor platform.

Control of all pumps was performed from the LabView application via the sensorboard.

1. Mesoscale bioreactor platform comprising:
two or more liquid reservoirs in fluid communication with a culture chamber, which chamber is in fluid communication with an exit, wherein the platform having a device allowing the chamber to be perfused with a flow of liquid from one or more of the liquid reservoirs.

2. Mesoscale bioreactor platform according to claim 1, wherein the culture chamber is accessible physically via a closable member or an elastic membrane.

3. Mesoscale bioreactor platform according to claim 2, wherein the closable member comprises a hinged lid or a sliding lid.

4. Mesoscale bioreactor platform according to claim 2, wherein the elastic membrane is made from a material having self-sealing capability.

5. Mesoscale bioreactor platform according to claim 1, wherein said device allowing the culture chamber to be perfused comprises a flexible region allowing the volume of the reservoir to be adjusted.

6. Mesoscale bioreactor platform according to claim 1, wherein said device allowing the culture chamber to be perfused comprises an air inlet allowing an external connection to the reservoir which air inlet comprises a filter with a pore size of around 0.1 μm to around 0.5 μm.

7. Mesoscale bioreactor platform according to claim 1, wherein the fluid communication between the two or more reservoirs and the culture chamber is provided by channels.

8. Mesoscale bioreactor platform according to claim 1, wherein the bioreactor platform comprises multiple culture chambers arranged in one or more groups of serially connected culture chambers.

9. Mesoscale bioreactor platform according to claim 1, wherein the culture chamber of the bioreactor platform comprises a scaffold supporting cellular growth.

10. Control unit for a mesoscale bioreactor platform according to claims 1, comprising a device configured to induce a flow rate of the liquid from the two or more reservoirs to the culture chamber.

11. Control unit according to claim 10, wherein said device configured to apply a positive relative pressure to said reservoirs and/or applying a negative relative pressure to said exit.

12. Control unit according to claim 11, wherein said device comprises a pump suited for pumping gases to the air inlets of the bioreactor platform and/or a liquid pump in fluid communication with the exit of the bioreactor platform.

13. Control unit according to claim 11, wherein said device comprises a pump, the pump being one of a piston pump, a syringe pump, a membrane pump, or a diaphragm pump, suited for pumping gases.

14. Control unit according to claim 13 further comprising a gas supply of a gas composition comprising 2-10% CO₂ and/or 2-20% O₂.

15. Control unit according to claim 11, wherein said device configured to apply a negative relative pressure to said exit comprises a pump for aspirating liquids, the pump being one of a peristaltic pump, a piston pump, a syringe pump, a membrane pump, a diaphragm pump, a gear pump, or a microannular gear pump.

16. Control unit according to claim claim 10, further comprising at least one sensor to measure pH, dissolved oxygen (O₂), carbon dioxide (CO₂), glucose, nutrients, vitamins, metabolites, flow velocity, temperature, optical density, a fluorescent signal, specific proteins or enzymes, or DNA's or RNA's.

17. Control unit according to any of claims 10 further comprising a light source, the light source being one of a light emitting diode (LED), a light bulb, a mercury lamp, or filters and a photodetector.

18. Control unit according to claim 10 further comprising a digital or an optical microscope.

19. Control unit according to claim 10 further comprising a temperature regulation system comprising a metal block shaped to house the bioreactor platform which block comprises a coil of an electrically conductive wire, a peltier element, or tubes for a heating and/or cooling liquid.

20. Control unit according to claim 10 comprising a compartment surrounding the mesoscale bioreactor platform, which compartment configured to be supplied with gases to create a laminar air flow around the bioreactor platform.

21. Control unit according to claim 16, further comprising a data processing unit to collect signals from the sensor and using the information from the collected signals for controlling the flow rate from each of the two or more reservoirs, the temperature, the gas composition and/or the laminar air flow.

22. System for culturing biological cells, comprising a mesoscale bioreactor platform according to claim 1 and a control unit, wherein the mesoscale bioreactor platform is a cartridge which fits into the control unit.

23. System according to claim 22, wherein the mesoscale bioreactor further comprises a radio frequency identification (RFID)-tag, and wherein the control unit comprises a sensor to read the RFID-tag.

24. Method of culturing a biological cell comprising:
providing a mesoscale bioreactor platform according to claim 1;
providing a control unit;
applying different culture media to each of the reservoirs;
providing a biological cell to the culture chamber;
perfusing the culture chamber with medium from one of or a combination of the reservoirs;
changing the medium from one to another reservoir or changing the combination of media from more reservoirs to match the requirements of the biological cell.

25. Method of culturing a biological cell according to claim 24, wherein the medium or a combination of media is changed based on a predetermined chronological series of events.

26. Method of culturing a biological cell according to claim 24, wherein the medium or a combination of media is changed based on signals collected from sensors in the control unit.

27. Method of culturing a biological cell according to claim 24, wherein the biological cell is a mammalian cell.

28. Method of culturing a biological cell according to claim 24, wherein the biological cell is a microbial cell.

29. Method of culturing a biological cell according to claim 27, wherein the mammalian cell is a stem cell or a cell of the immune system.

30. Method of culturing a biological cell according to claim 27, wherein the mammalian cell is an unfertilised or a fertilised oocyte.

31. The method of claim 29, wherein the mammalian cell is selected from the group consisting of monocyte, dendritic cell, or a T-cell.
